(19) United States
(12) Patent Application Publication
(10) Pub. No.: US 2023/0398330 A1
(43) Pub. Date: Dec. 14, 2023

(54) PEEL-AWAY INTRODUCER SHEATH HAVING AN ADJUSTABLE DIAMETER AND METHOD OF USE

(71) Applicant: KARDION GMBH, Stuttgart (DE)

(72) Inventors: Marvin Mitze, Stuttgart (DE); Leon Wenning, Stuttgart (DE); Johannes Bette, Balingen (DE); Mark Stuart Leung, Duncan, British Columbia (CA); Hans Christof, Unterensingen (DE)

(21) Appl. No.: 18/020,062

(22) PCT Filed: Aug. 4, 2021
(86) PCT No.: PCT/US2021/071105

§ 371 Date: Feb. 6, 2023

Related U.S. Application Data
(60) Provisional application No. 63/063,006, filed on Aug. 7, 2020.

(51) Int. Cl. A61M 25/06; A61M 25/00

(52) U.S. Cl. CPC A61M 25/0668; A61M 25/0023; A61M 2025/0024; A61M 25/005; A61M 25/0068; A61M 25/0097

(57) ABSTRACT

Peel-away introducer sheath (100) having an adjustable diameter. The present disclosure is directed generally to introducer sheaths used in endoluminal catheterization for medical treatments. More particularly, the present disclosure is directed towards introducer sheaths that are expandable and can be peeled apart.

### CROSS-REFERENCE TO RELATED APPLICATIONS

This application claims the priority benefit of U.S. Provisional Patent Application No. 63/063,006, filed Aug. 7, 2020, the entire disclosure of which is hereby incorporated by reference herein in its entirety.

### TECHNICAL FIELD

The present disclosure is directed generally to introducer sheaths used in endoluminal catheterization for medical treatments. More particularly, the present disclosure is directed towards introducer sheaths that are expandable and can be peeled apart.

### BACKGROUND

Introducer sheaths are used in medical interventions that employ access to a natural cavity or lumen of the patient, such as the vessels of the vascular system, as a method of gaining access to a target region of the patient's body where material may be delivered, removed or an intervention can be performed. In a typical endovascular procedure, for example, access to the vasculature of the patient involves making a small percutaneous incision or puncture with a scalpel or introducer needle and delivering endovascular devices, such as catheters, into the lumen. An introducer sheath plays an important role in the procedure to protect the access puncture from excessive shear or frictional forces, to protect medical devices as they are delivered into the vasculature, to provide structural support, or to guide the delivery of a medical device through a desired luminal pathway. Compared to alternative approaches for accessing said target regions, such as open surgery, an endovascular approach or other approach utilizing an introducer sheath often has significant benefits such as reduced risk of infection or iatrogenic injury and quicker recovery.

A vast array of endoluminal medical devices has been developed for such less invasive approaches having dimensions and other features allowing them to be advanced into the natural lumens. Often such medical devices are elongate and tubular having outer diameters that are smaller than the smallest natural lumen through which they are intended to pass through to facilitate delivery, avoid injury, and to minimize interruption of lumen function, such as blood flow in the case of an endovascular procedure.

More recently, solutions have been explored for endoluminal delivery of devices that, including an introducer sheath, have a diameter that is large enough to occlude the lumen or even distend it. A device requiring such solutions may need to be larger as a result of design constraints. Furthermore, such solutions could permit patients, otherwise excluded from an endoluminal procedure due to narrow lumens or calcified vessels, to receive treatment. Challenges of endoluminal delivery of such larger devices may include disrupting a function of the luminal organ such as occlusion of the lumen or application of forces that are intolerable to the tissue. For example, for an endovascular procedure involving delivery of a medical device and an associated introducer sheath into a blood vessel having an inner diameter that is close to or even smaller than the maximum outer diameter of the introduced devices may result in occlusion of blood flow risking ischemia or injury to the blood vessel due to shear or distension. There is a need for solutions for delivering large devices with an endoluminal approach so the safety and procedural benefits of endoluminal approaches over surgery in general can be applied to larger devices.

One previously proposed solution includes peel away introducer sheaths. A peel away introduce sheath may have a large diameter suitable for delivering the large medical device into the lumen but may occlude the lumen while in place. Once the large medical device is delivered, the peel-away introducer sheath may be retracted out of the body where it is positioned over a shaft of the large medical device, which may have a smaller diameter than the medical device itself. The shaft may be connected to a larger handle obstructing complete removal of the introducer sheath past the handle. The peel-away introducer sheath is removed from the shaft by peeling it apart thus opening the lumen of the introducer sheath. The peel-away introducers are adapted to be peeled apart by providing grab tabs connected to separate parts of the sheath held together by one or more seams that tear when shear force is applied by pulling the grab tabs apart. Optionally, a replacement introducer having a smaller diameter can be advanced into the lumen following removal of the tear away sheath if necessary.

Another previously proposed solution includes expandable introducer sheaths that have a lumen diameter and outer diameter that can be stretched, for example along a longitudinal seam or fold, from a neutral state to an expanded state to temporarily accommodate passage of a medical device then return to the neutral state, which may have a suitable diameter that minimizes invasiveness, risk of tissue injury, or obstruction of the natural lumen.

Expandable introducer sheaths and peel-away introducer sheaths are valuable tools for endoluminal medical interventions. However, there remains a need for introducer sheath designs that allow passage of medical devices having dimensions that may occlude or distend the lumen in which the introducer sheath is placed, that further improves function and versatility.

### SUMMARY

This disclosure is related to methods, devices, and systems for introducer sheaths that are expandable, or that may be peeled apart, or both. The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for introducer sheaths.

The following disclosure describes non-limiting examples of some embodiments of introducer sheaths. For instance, other embodiments of the disclosed systems and methods may or may not include the features described herein. Moreover, disclosed advantages and benefits can apply only to certain embodiments and should not be used to limit the disclosure.

A 1^{st} aspect of the disclosure relates to an introducer sheath, including: an elongate, flexible tubular body having a proximal end, a distal end, and a side wall defining a central lumen; a hub on the proximal end; and at least one split line extending axially along the side wall; wherein at least a portion of the tubular body is selectively radially outwardly expandable in response to radially outward force applied against the side wall.

A 2^{nd} aspect relates to an introducer sheath as in aspect 1, wherein the side wall has a neutral state in which the lumen has a first inside diameter and an expanded state in which the inside diameter is selectively increased locally in response to a medical device having a second diameter larger than the first inside diameter passing axially through the lumen.

A 3^{rd} aspect relates to an introducer sheath as in aspect 2 wherein the inside diameter in the neutral state is within the range of from about 6F to about 10F.

A 4^{th} aspect relates to an introducer sheath as in aspect 3 wherein the inside diameter in the neutral state is about 8F.

A 5^{th} aspect relates to an introducer sheath as in aspect 2 wherein the lumen is only in the expanded state in a selectively expanded portion of the tubular body that is under radial force due to the presence of the medical device.

A 6^{th} aspect relates to an introducer sheath as in aspect 5 wherein the selectively expanded portion advances axially along the tubular body in response to axial movement of the medical device.

A 7^{th} aspect relates to an introducer sheath as in any preceding aspects 5 or 6 wherein the inside diameter of the expanded portion is at least about 105% of the inside diameter of an adjacent section of the tubular body in the neutral state.

A 8^{th} aspect relates to an introducer sheath as in any preceding aspects 5 or 6 wherein the inside diameter of the expanded portion is at least about 115% of the inside diameter of an adjacent section of the tubular body in the neutral state.

A 9^{th} aspect relates to an introducer sheath as in any preceding aspects 1 to 8, wherein the side wall is radially outwardly expandable by stretching in response to the presence of the medical device.

A 10^{th} aspect relates to an introducer sheath as in any preceding aspects 1 to 9, wherein the side wall is radially outwardly expandable by unfolding in response to the presence of the medical device.

An 11^{th} aspect relates to an introducer sheath as in any preceding aspects 1 to 10, wherein the split line includes a weakening along which the side wall can split to expose the lumen.

A 12^{th} aspect relates to an introducer sheath as in any preceding aspects 1 to 11, wherein the split line includes a first edge of the sidewall and a second edge of the sidewall held together by a removable pull element.

A 13^{th} aspect relates to an introducer sheath as in aspect 12, wherein the first edge includes a first plurality of loops, the second edge includes a second plurality of loops, and until it is removed, the pull element holds the first and second edges together.

A 14^{th} aspect relates to an introducer sheath as in any preceding aspects 1 to 13, further includes a homeostasis valve in the hub.

A 15^{th} aspect relates to an introducer sheath as in any preceding aspects 1 to 14, wherein the hub includes an annular side wall having a closed, annular configuration and an open configuration including a longitudinal opening in the hub sidewall.

A 16^{th} aspect relates to an introducer sheath as in aspect 15, wherein the longitudinal opening is formed by removing an axial portion of the sidewall.

A 17^{th} aspect relates to an introducer sheath as in aspect 15, wherein the longitudinal opening is formed by splitting the hub axially into two pieces.

An 18^{th} aspect relates to an introducer sheath as in aspect 15, wherein the longitudinal opening is formed by splitting the hub axially into two halves.

A 19^{th} aspect relates to an introducer sheath as in aspect 17, further including a pull tab on at least one of the two pieces.

A 20^{th} aspect relates to an introducer sheath as in aspect 17, further including a removable retainer for retaining the two pieces together.

A 21^{st} aspect relates to an introducer sheath as in aspect 18, further including a pull tab on each half.

A 22^{nd} aspect relates to an introducer sheath as in any preceding aspects 1 to 21, further including a locking mechanism on the hub, configured to engage an accessory device.

A 23^{rd} aspect relates to an introducer sheath as in aspect 22, wherein the accessory device includes a dilator.

A 24^{th} aspect relates to an introducer sheath as in aspect 22, wherein the accessory device includes an insertion tool.

A 25^{th} aspect relates to an introducer sheath as in any preceding aspects 1 to 24, wherein the tubular body includes a radially expandable structural layer and an expandable membrane.

A 26^{th} aspect relates to an introducer sheath as in aspect 25, wherein the structural layer includes braided, woven, or knitted fibers.

A 27^{th} aspect relates to an introducer sheath as in aspect 26, wherein the fibers are superelastic Nitinol or polymer.

A 28^{th} aspect relates to an introducer sheath as in any preceding aspects 26 or 27, wherein the fibers have a thickness in a range of 0.003″ to 0.010″.

A 29^{th} aspect relates to an introducer sheath as in any preceding aspects 26 or 27, wherein the braided, woven, or knitted fibers include a braid angle that is greater in the expanded state than in the neutral state.

A 30^{th} aspect relates to an introducer sheath as in any preceding aspects 25 to 29, wherein the structural layer overlaps on itself for at least a portion of a circumference of the tubular body.

A 31^{st} aspect relates to an introducer sheath as in aspect 25, wherein the structural layer includes a laser cut tube.

A 32^{nd} aspect relates to an introducer sheath as in aspect 31, wherein the laser cut tube includes a plurality of longitudinal members aligned parallel to an axis of the tubular body, the longitudinal members connected to adjacent longitudinal members by a plurality of angled cross members.

A 33^{rd} aspect relates to an introducer sheath as in any preceding aspects 1 to 32, wherein the at least one split line includes a polymer strip.

A 34^{th} aspect relates to an introducer sheath as in aspect 33 in combination with aspect 25, wherein the structural layer is connected to the polymer strip.

A 35^{th} aspect relates to an introducer sheath as in any preceding aspects 1 to 34, further including an expandable atraumatic distal tip on the distal end.

A 36^{th} aspect relates to an introducer sheath as in aspect 35 in combination with aspect 25, wherein the expandable atraumatic distal tip is made from the same material as the expandable membrane.

A 37^{th} aspect relates to an introducer sheath as in any preceding aspects 35 or 36, wherein the expandable atraumatic distal tip includes at least one folded pleat.

A 38^{th} aspect relates to an introducer sheath as in any preceding aspects 35 to 37, wherein the expandable atraumatic distal tip includes at least one wedge-shaped cut out.

A 39^{th} aspect relates to an introducer sheath as in any preceding aspects 35 to 38, wherein the expandable atraumatic distal tip includes a helical groove or threading.

A 40^{th} aspect relates to an introducer sheath as in any preceding aspects 35 to 39, wherein the expandable atraumatic distal tip includes an expanded configuration in a neutral state.

A 41^{st} aspect relates to an introducer sheath as in aspect 40, wherein the expanded configuration of the expandable atraumatic distal tip includes a funnel shape.

A 42^{nd} aspect relates to an introducer sheath as in aspect 41 in combination with aspect 23, wherein the dilator is configured to releasably hold the expandable atraumatic distal tip in a compressed state.

A 43^{rd} aspect relates to an introducer sheath as in aspect 42, wherein the dilator includes a handle with an actuator configured to release the expandable atraumatic distal tip from the compressed state.

A 44^{th} aspect relates to an introducer sheath as in any preceding aspects 1 to 43, further including at least one longitudinal support wire that limit elongation of the tubular body.

A 45^{th} aspect relates to an introducer sheath as in any preceding aspects 1 to 44, further including an inner surface that is at least one of hydrophilic, micropatterned, or lubricious.

A 46^{th} aspect relates to an introducer sheath as in aspect 45, wherein the inner surface includes PTFE.

A 47^{th} aspect relates to an introducer sheath as in any preceding aspects 1 to 46 in combination with aspect 21, wherein the first pull tab on the first half wraps around at least a portion of the hub connecting the first half to the second half.

A 48^{th} aspect relates to an introducer sheath as in aspect 47, wherein the first pull tab removably mates with the second pull tab on the second half.

A 49^{th} aspect relates to an introducer sheath as in any preceding aspects 1 to 48, wherein the hub includes a cap.

A 50^{th} aspect relates to an introducer sheath as in aspect 49, wherein the cap is configured to hold the hub together.

A 51^{st} aspect relates to an introducer sheath as in any preceding aspects 1 to 50, wherein the central lumen has a maximum expansion limit.

A 52^{nd} aspect relates to an introducer sheath as in aspect 51, wherein the maximum expansion limit is in a range of 19F to 27F.

A 53^{rd} aspect relates to an introducer sheath as in any preceding aspects 51 to 52, in combination with aspect 25, wherein the maximum expansion limit is defined by an elongation factor of the membrane.

A 54^{th} aspect relates to an introducer sheath as in aspect 53, wherein the elongation factor is in a range of 1.25 to 4.33.

A 55^{th} aspect relates to an introducer sheath as in aspect 53, wherein the elongation factor is in a range of 1.875 to 2.375.

A 56^{th} aspect relates to an introducer sheath as in any preceding aspect 1 to 55, further including a cutting tool configured to cut the split line of the tubular body.

A 57^{th} aspect relates to an introducer sheath as in aspect 56, wherein the cutting tool includes a blade, a tongue, and a handle, wherein the blade is located between the tongue and handle.

A 58^{th} aspect relates to an introducer sheath as in any preceding aspect 56 to 57, wherein the blade is held at an obtuse angle in relation to an axis of the tubular body.

A 59^{th} aspect relates to an introducer sheath as in any preceding aspects 56 to 58 in combination with aspect 49, wherein the cutting tool includes a tab configured to mate with the cap.

A 60^{th} aspect relates to an introducer sheath as in any preceding aspects 57 to 59, wherein the handle includes a textured surface.

A 61^{st} aspect relates to an introducer sheath as in any preceding aspects 57 to 60, wherein the tongue includes a curved surface having a radius of curvature that is within 5% of a radius of curvature of the tubular body.

A 62^{nd} aspect relates to a system including an introducer sheath of any preceding aspects 1 to 61, further including a medical device configured to be advanced through the introducer sheath, wherein the medical device includes a proximal end, the introducer sheath includes a distal end, and the proximal end of the medical device is configured to increase a diameter of the distal end of the introducer sheath when the medical device is retracted from the introducer sheath.

A 63^{rd} aspect relates to a system as in aspect 62, wherein the proximal end of the medical device is configured to increase the diameter of the distal end of the introducer sheath when the medical device is rotated about its axis.

A 64^{th} aspect relates to a system as in aspect 63, wherein the proximal end of the medical device includes a helical structure configured to engage with a structure on the distal end of the introducer sheath and to increase a diameter of the distal end of the introducer sheath when the medical device is rotated while retracted.

A 65^{th} aspect relates to a system as in aspect 63, wherein the distal end of the introducer sheath includes a helical structure configured to engage with a structure on the proximal end of the medical device and to increase a diameter of the distal end of the introducer sheath when the medical device is rotated while retracted.

A 66^{th} aspect relates to a method of introducing a medical device through an introducer sheath, including the steps of: providing an expandable, peel away introducer sheath having a proximal end, a distal end, and a tubular side wall defining a central lumen selectively enlargeable from a relaxed inside diameter to an enlarged inside diameter; positioning the introducer sheath into a vessel of a patient while in the relaxed inside diameter configuration; providing a catheter carrying a medical device which has an outside diameter that is larger than the relaxed inside diameter; advancing the medical device distally through the lumen causing an expansion zone of the side wall in the vicinity of the medical device to momentarily reach the enlarged diameter to accommodate passage of the medical device; and thereafter splitting the introducer sheath axially to create a longitudinal opening and expose the lumen to permit lateral removal of the introducer sheath from the catheter.

A 67^{th} aspect relates to a method as in aspect 66, wherein the expansion zone progressively advances distally along the tubular side wall in response to distal advance of the medical device.

A 68^{th} aspect relates to a method as in aspect 67, wherein the medical device includes a mechanical circulatory support device.

A 69^{th} aspect relates to a method as in aspect 66, wherein splitting the introducer includes pulling on at least one pull tab to create the longitudinal opening.

A 70^{th} aspect relates to a method as in aspect 66, further including the step of coupling an insertion tool to the proximal end prior to the advancing step.

A 71^{st} aspect relates to a method as in aspect 69, wherein splitting the introducer includes pulling on two opposing pull tabs to axially split the sheath into two pieces.

A 72^{nd} aspect relates to a method as in aspect 70, wherein the insertion tool includes an extension tube configured to be advanced into a patient.

A 73^{rd} aspect relates to a method as in aspect 72, further including a step of advancing the extension tube of the insertion tool into the vessel of the patient following the removal of the introducer sheath from the catheter.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawing, can be arranged, substituted, combined, and designed in a wide variety of different configurations, all of which are explicitly contemplated and make part of this disclosure.

FIG. 1 is a schematic illustration of a peel away expandable introducer sheath and dilator inserted into an artery over a guidewire.

FIG. 2 is a cross sectional view of a peel away expandable introducer sheath and dilator.

FIG. 3 is a cross sectional view of a portion of an elongated tube of a peel away expandable introducer sheath in a neutral state.

FIG. 4 is a cross sectional view of a portion of an elongated tube of a peel away expandable introducer sheath in an expanded state.

FIG. 5 is a schematic illustration of a peel away expandable introducer sheath longitudinally separated into two parts to release a medical device shaft.

FIG. 6 is a schematic illustration of an example introducer handle separated longitudinally.

FIG. 7A is a schematic illustration of an alternative example introducer.

FIG. 7B is a schematic illustration of an alternative example introducer.

FIG. 8A is a schematic illustration of another alternative example introducer wherein the handle remains as one piece and the elongated tube tears apart and is removed from the handle.

FIG. 8B is a schematic illustration of the introducer of FIG. 8A with an insertion tool positioned in the introducer's hub.

FIG. 9A is a schematic illustration of an elongated tube of an introducer, made with braided or woven fibers, having an expanded section.

FIGS. 9B, 9C, and 9D are schematic illustrations of examples of seams on an elongated tube made with braided or woven fibers.

FIGS. 9E, 9F, 9G, and 9H are cross sectional schematic illustrations of examples of introducers made with braided or woven fibers.

FIG. 10A is a schematic illustration of an alternative embodiment of an elongated tube of an introducer made with longitudinal members connected by angular cross members.

FIG. 10B is a cross sectional view of the elongated tube of FIG. 10A.

FIGS. 11A and 11B are schematic illustrations of another alternative embodiment of an elongated tube of an introducer made with a polymer support layer and stretchable membrane layer.

FIGS. 12A, 12B, 12C, and 12D are schematic illustrations of embodiments of an expandable distal end of an introducer.

FIG. 12E is a schematic illustration of another embodiment of an expandable distal end of an introducer having a helical threading adapted to mate with a helical groove on a medical device to facilitate retraction of the medical device into the introducer.

FIG. 12F is a schematic illustration of the introducer and medical device of FIG. 12E, wherein the medical device is being retracted into the distal end of the introducer.

FIGS. 13A, 13B and 13C are schematic illustrations of another embodiment of an introducer's expandable distal end and a dilator adapted to manipulate the shape of the distal end.

FIGS. 14A, 14B and 14C are schematic illustrations of another embodiment of an introducer's expandable distal end and a dilator adapted to manipulate the shape of the distal end.

FIG. 15 is a partial isometric view of another embodiment of an introducer.

### DETAILED DESCRIPTION

The disclosure herein is related to peel-away introducer sheaths having expandable or adjustable lumen diameters, kits comprising the introducer sheath and other tools for use and methods of use. The following detailed description is directed to certain specific embodiments. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings.

The embodiments and features of introducer sheaths may be applicable or adapted for use in most endoluminal interventions, for example for passage of a medical device through natural anatomical lumens such as bronchi, urethras, intestines, ducts or blood vessels. For illustrative purposes, detailed descriptions of exemplary embodiments are focused on endovascular procedures and in particular for delivery of mechanical circulatory support (MCS) devices into an artery such as a femoral artery. However, it is to be understood that components and features disclosed herein may be combined with one another in any suitable way and may be adapted for passage of other medical devices or with other endoluminal interventions.

Furthermore, systems, devices and methods for delivery, maintenance and retraction of a mechanical circulatory support device using peel-away expandable introducer sheaths are also disclosed.

A peel-away introducer sheath having an adjustable lumen and outer diameter can incorporate benefits of both peel-away introducer sheaths and expandable introducer sheaths while providing versatility to use different features of the sheath. For example, a peel-away expandable introducer sheath may be used as a peel-away introducer sheath, as an expandable introducer sheath, or as both depending on a physician's familiarity or preference. Furthermore, the introducer may provide adaptability for a physician to use the features of the introducer sheath depending on the situation at hand. The adaptability and versatility may facilitate faster procedure time or improvement of patient safety.

Another benefit is that such an introducer sheath may be used with a variety of devices having different sizes, which can improve supply efficiency. For example, a medical device supplier may offer a plurality of mechanical circulatory support devices having maximum diameters in a range of 14F to 21F (e.g., in a range of 14F to 18F) and minimum diameters in a range of 8F to 10F, and the supplier may provide a single model of expandable peel-away introducer sheath, optionally with each of the plurality of devices in a kit, that is capable of passage of the plurality of devices while conforming to a minimal diameter.

A peel-away expandable introducer sheath, like conventional introducer sheaths, may be placed in a natural lumen, such as a blood vessel (e.g., femoral artery), through an orifice or aperture, such as an arteriotomy, and provide an access route into the natural lumen from outside of the body while protecting tissue and devices delivered through the introducer sheath. Also, like conventional introducer sheaths, a peel-away expandable introducer sheath may have features such as hemostasis valves, flushing ports, suture eyelets, and be used with a dilator. However, peel-away expendable introducer sheath can temporarily expand in diameter to conform to and accommodate delivery of a medical device and contract towards a neutral state decreasing in diameter when radial force applied to the inner lumen is removed. For example, a medical device may include an elongate shaft with a component mounted to the distal end of the shaft wherein the component has a larger diameter then the shaft. Passage of the larger component through the introducer sheath may cause the sheath to expand in diameter and when the larger component passes out of the introducer the smaller shaft can remain connected to the larger component and slidably engaged within the introducer allowing the introducer diameter to reduce in size. Furthermore, the peel-away expandable introducer may be retracted from the body, with the medical device shaft still contained in the introducer's lumen, and be removed from the medical device shaft by tearing or peeling a longitudinal opening along the length of the introducer.

### General Features of a Peel Away Expandable Introducer

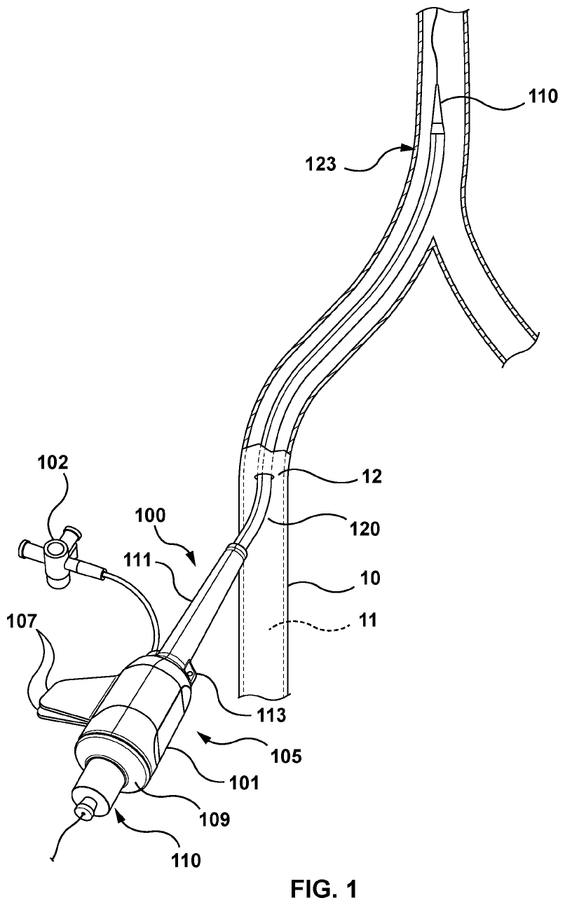

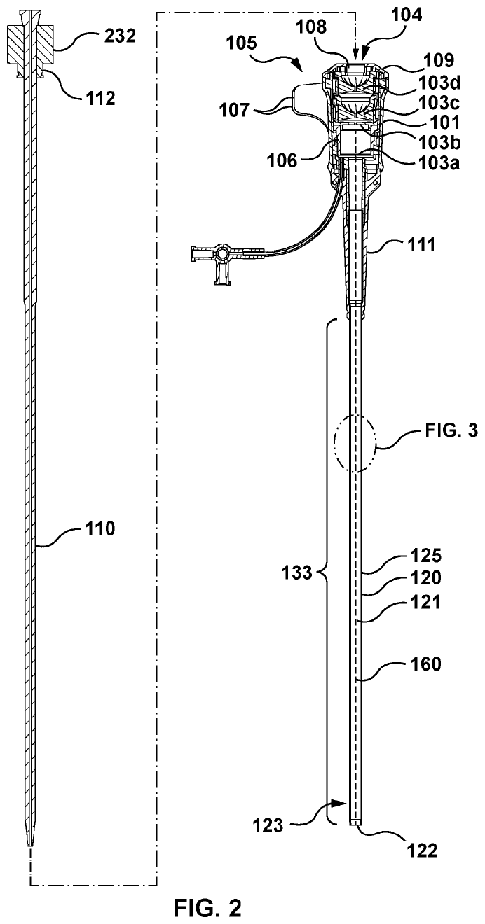

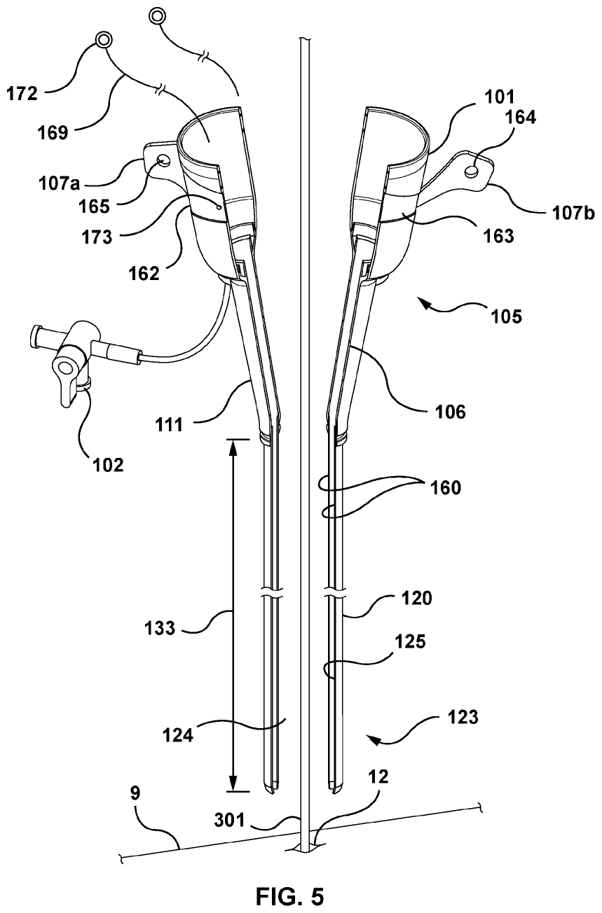

Referring to FIG. 1 and FIG. 2, a peel-away expandable introducer sheath 100 may include a proximal end 105 and a distal end 123, a hub 101 at the proximal end intended to remain exterior to the patient's body 9, an elongated tube 120 intended to be inserted into a natural lumen (e.g., through an arteriotomy 12 into a lumen 11 of an artery 10), and an introducer lumen 121 passing through the hub and elongated tube and exiting at a proximal opening 104 in the hub and a distal opening 122 at the distal end of the elongated tube. The introducer lumen is defined by a wall 125 of the elongated tube and a wall 106 of the hub, both of which are adapted to be opened or peeled apart in use along the full length of the introducer creating a longitudinal opening 124 to allow contents (e.g., a medical device shaft 301) held within the introducer lumen to be released through the longitudinal opening 124 as shown in FIG. 5. The elongated tube 120 is adapted to have a neutral state and an expanded state.

### Introducer Hub

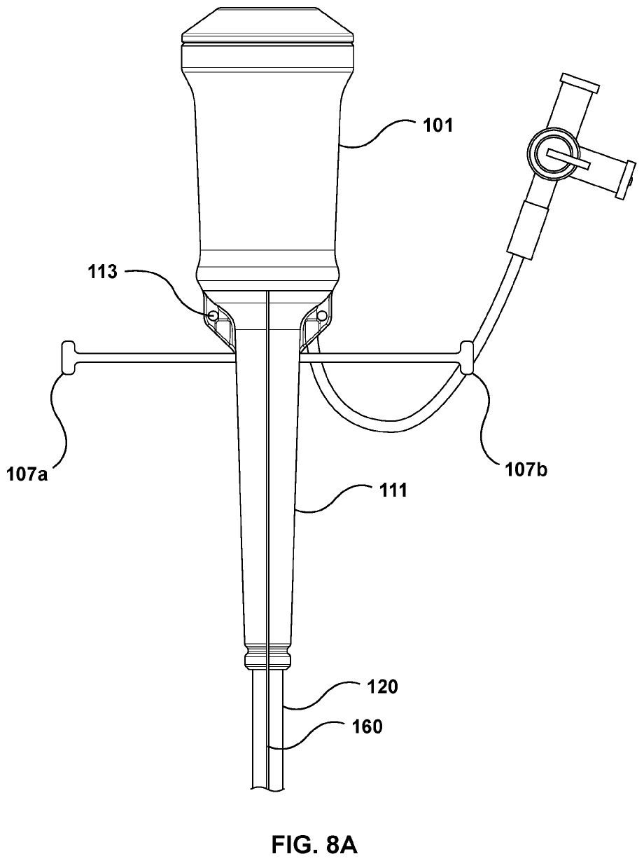

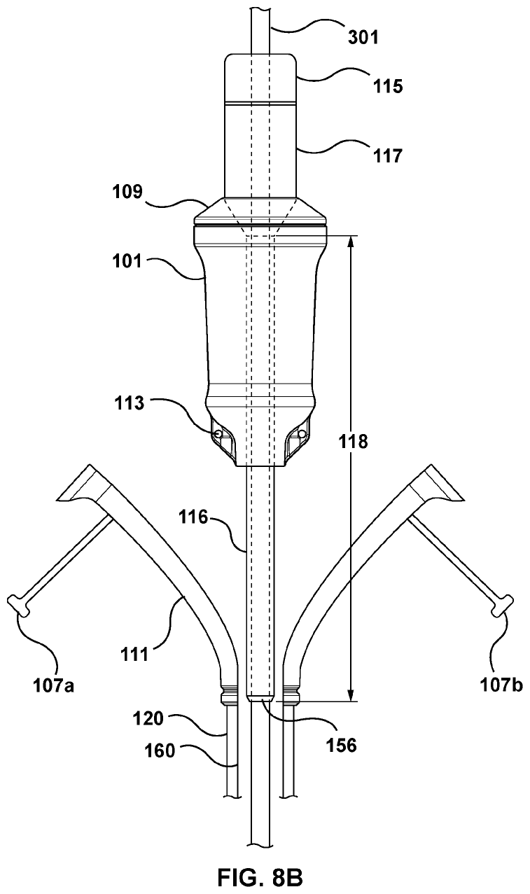

Referring to FIG. 2, the hub 101 may be provided in a closed configuration and be openable (see FIG. 5) along one or more seams 160 to release a medical device 301 from the lumen 121 through a longitudinal opening 124. The pull tab(s) 107 may be located on the hub wherein each tab is connected to opposing sides of the introducer such that force applied to pull the pull tabs apart from one another applies a shear to the seam pulling it apart. The hub may optionally contain one or more hemostasis valves 103 that allow delivery of tools such as guidewires, catheters and other medical devices through the hub while maintaining a fluid tight seal. For example, a peel away expandable introducer sheath intended for delivery of an MCS device may have multiple hemostasis valves for 0″ (103a), an 0.018″ guidewire (103b), a 0.035″ guidewire (103c), and a 5F or 6F diagnostic catheter (103d). The MCS device may be larger, for example having a diameter in a range of 14F to 18F, and be delivered through the multiple hemostasis valves using an insertion tool that spreads the hemostasis valves open to reduce insertion force and to protect the medical device (e.g., MCS device) from forces applied by the hemostasis valves. An insertion tool 115 (shown in FIG. 8B) may for example have a handle 117 that mates (e.g., locks, threads, click, friction fits) into the introducer handle 101 (e.g., handle cap 109), an elongated tube 116 with a length 118 that is at least long enough to extend into the strain relief 111 when the handle 117 is mated with the introducer hub 101. The insertion tool's elongated tube 116 may be flexible enough to bend but have sufficient rigidity or hoop strength to resist compression by the hemostasis valves. A lumen 119 passes through the insertion tool through which a medical device may be delivered. Optionally, the insertion tool may also have a hemostasis valve in its lumen 119 adapted to seal against the medical device 300 or its shaft 301. Optionally, the insertion tool's elongated tube 116 may be adapted to be used as an introducer sheath, as is described later with respect to FIG. 8B, for example, having an atraumatic distal tip, which may have a soft durometer or taper or flexible expandable diameter. Optionally, an insertion tool dilator may be used to advance the extension tube 116 of the insertion tool into a patient's lumen (e.g., arteriotomy). An insertion tool dilator may be an elongated tube with a tapered distal end that is adapted to be assembled on a medical device shaft 301 and advanced through a lumen of the insertion tool's extension tube 116 to position the tapered distal end at the distal opening of the extension tube 116. For example, the insertion tube's dilator may be provided in two halves or with a longitudinal opening for a user to assemble over a medical device shaft 301. The dilator may have an inner lumen sized to slidably accommodate a medical device shaft 301 and an outer diameter sized to be slidably accommodated by the insertion tool's extension tube 116. The tapered distal end may taper from the outer diameter to the inner lumen diameter. In use the dilator may be pulled out of the insertion tool before removing the medical device from the patient so medical device can fit through the insertion tool's extension tube 116. The hub 101 may optionally have a locking mechanism 108 on its proximal end 105, for example as part of a cap 109, configured to mate with a locking mechanism 112 on the dilator 110 or insertion tool. The locking mechanism 108, 112 may be for example a snap fit, friction fit, threaded or O-ring mating mechanism. The hub may also have a flushing port 102 that is attachable to a syringe and used to flush air from the lumen 121 prior to delivery into the body. The hub may also have a strain relief 111 that functions to hold the proximal end of the elongated tube to the handle and allow bending while avoiding kinking particularly where the flexible elongated tube 120 meets the rigid hub 101. The hub may also have suture eyelets 113 for suturing the hub to the patient's skin, in particular to hold the introducer in place during longer procedures.

### Neutral State of Elongated Tube

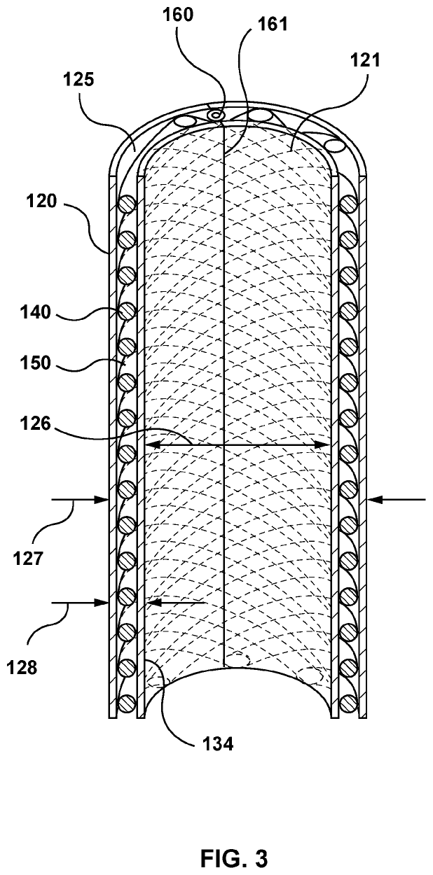

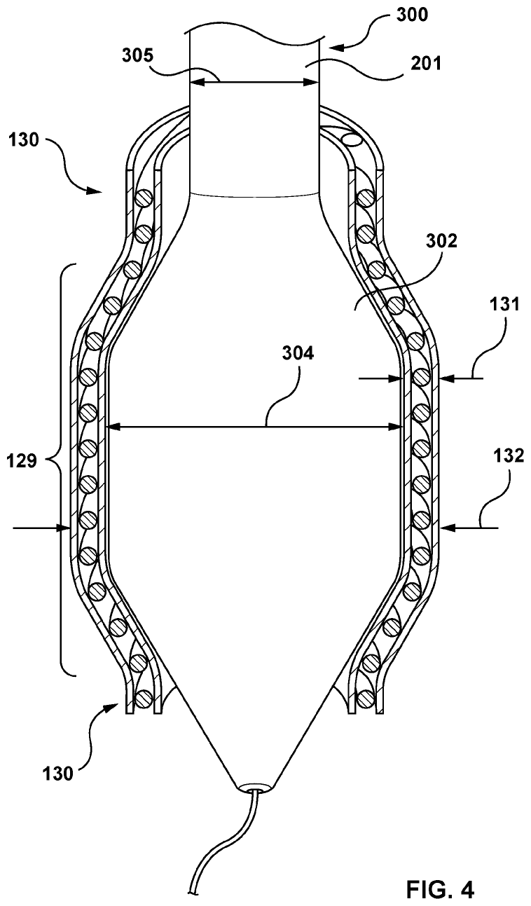

A cross-sectional close-up view of an embodiment of the elongated tube 120 in its neutral state is shown in FIG. 3. The wall of the elongated tube 125 has a neutral state having a neutral state inner diameter 126, as shown in FIG. 3, and may be temporarily radially expanded when radial force is applied to the inner surface of the wall, for example when a large component 302 of a medical device having a diameter 304 that is larger than the neutral state diameter 126 is passed through the introducer lumen 121 as shown in FIG. 4. The neutral state of the elongated tube 120 may have an inner diameter 126 adapted to slidably engage components used in a catheterization procedure such as guidewires, dilators, diagnostic catheters, treatment catheters or a shaft of a medical device wherein radial expansion may not be necessary. For example, a peel-away expandable introducer sheath intended for transfemoral artery delivery of an MCS device may have an inner diameter 126 in a range of 6F to 1° F. (preferably 8F). In its neutral state the elongated tube shall be adapted to be kink resistant and have sufficient compressive and tensile strength to allow devices to be slidably advanced or retracted through the inner lumen and to be advanced through an artery or other natural lumen. The neutral state of the elongated tube 120 may have wall thickness 128 in a range of 0.3 to 0.5 mm and an outer diameter 127 equal to the inner diameter 126 plus two times the wall thickness 128. The neutral state outer diameter 127 may be chosen to allow sufficient functioning of the lumen through which the elongated tube is positioned in. For example, the outer diameter 127 of an expandable peel-away introducer sheath intended for transfemoral artery delivery of an MCS device may have a neutral state outer diameter 127 in a range of 6.5F to 12F, preferably in a range of 8F to 11F, which may apply minimal or no radial force to the artery wall and may allow sufficient blood flow through the femoral artery for the intended duration of implantation (e.g., several hours to several days) to avoid ischemia of the limb. Optionally, during delivery a dilator 110 (see FIG. 2) having longitudinal rigidity may help to maintain the length of the elongated tube 120 to facilitate delivery into the patient's lumen (e.g., artery).

### Expanded State of Elongated Tube

The expanded state of the elongated tube 120, as shown in FIG. 4, may have an expanded section 129 that is expanded as a result of radial force applied to the inner wall by the advancement of a device (e.g., a MCS device 302) having a diameter that is larger than the neutral state inner diameter 126, and at least a portion of the remaining length 130 of the elongated tube 120 of which radial force is not applied may resemble the neutral state and have a neutral state outer diameter 127. For example, the inner diameter of the expanded section 129 may conform to the diameter 304 of the device 302. The expanded section 129 may move along the length of the elongated tube 120 in reaction to a position of the device 302. For example, the inner diameter of the expanded section 129 may accommodate devices having a diameter up to 26F (preferably up to 19F), which may allow delivery of both a 14F and 18F MCS device. The wall thickness 131 of the expanded section may be equal to or less than the neutral state wall thickness 128. In some embodiments the wall thickness may decrease with expansion of diameter for example due to stretching or elongation of materials. Consequently, the outer diameter 132 in the expanded section 129 is a function of the device diameter 304 and wall thickness 131. The elongated tube 120 optionally may have a maximum expansion limit to prevent injury to the blood vessel or natural lumen. For example, in the case of transfemoral artery positioning a maximum expansion limit may be in a range of 19F to 27F and be a function of materials and design of the elongated tube. For example, an elongated tube may be made with an elastically stretchable material having an elongation factor equal to the maximum outer circumference divided by the neutral state circumference. For an illustrative example of an introducer intended for delivery of an MCS device, the elongated tube 120 may have an expansion ratio of the maximum expanded inner diameter to neutral state inner diameter in a range of 10:8 (i.e., 1.25) to 26:6 (i.e., 4.33), preferably in a range of 15:8 (i.e., 1.875) to 19:8 (i.e., 2.375).

The elongated tube 120 may have a length 133 adapted to traverse the natural lumen to provide access to a target location. For example, an introducer used to deliver an MCS device through a femoral artery to reach past a femoral bifurcation may have a neutral state length in a range of 250 to 350 mm, preferably a minimum of 275 mm. In some embodiments the length 133 of the elongated tube may shorten as a result of expanding a portion of the elongated tube. In some embodiments the length 133 may be restricted from increasing beyond its neutral state by tension in longitudinal support wires 161, which in turn may prevent the neutral state inner diameter 126 of the lumen 121 from decreasing, which may facilitate passage of a medical device through the lumen 121.

### Further Elongated Tube Features

Referring to FIG. 3, the elongated tube 120 may be made with an expandable structural layer 140 and an expandable membrane 150 and have a seam 160. The expandable structural layer 140 may function to provide structural support to maintain a tubular shape, allow bending while resisting kinking over a bend radius of at least 10 cm, provide longitudinal strength in both compression and tension to allow the introducer to be advanced into a body lumen and pulled out of it, and to guide the delivery of a medical device through a desired lumen pathway. The expandable membrane 150 may function to seal the wall 125 of the elongated tube to prevent leakage of liquid such as blood, water, or saline through the wall for example, to allow air to be flushed from the lumen 121 prior to introduction into a body lumen or to prevent blood from leaking out of the lumen through the wall during use. The membrane may also function to protect tissue from injury by providing a smooth surface. Optionally, the elongated tube may have one or more (preferably two) longitudinal support wires to add longitudinal strength and limit elongation. Optionally, the elongated tube may further have an inner layer 134 that reduces friction and improves slidability between the inner layer and a medical device 302. Optionally, the elongated tube may further have an outer layer 135 that reduces friction and improves slidability between the outer layer and tissue of the body lumen. The inner layer and outer layer may be an additional material (e.g., PTFE) laminated to the membrane 150 or structural layer 140, or a coating or surface treatment (e.g., micropatterned surface) on the expandable membrane and may have hydrophilic or lubricious properties. Optionally, a material in the elongated tube wall 125 may be impregnated with an agent (e.g., antibacterial, antimicrobial) that may reduce a risk of infection particularly when in use for several days.

### Seam and Pull Tabs

The seam(s) 160 functions to hold the elongated tube 120 together in a tubular form and to be opened to create a longitudinal separation 124 along the length of the elongated tube to facilitate removal of a medical device 301 contained in the lumen 121. Separation of the seam may be facilitated by pulling on one or more (e.g., two) pull tabs 107 that may remove a connecting seam structure and/or apply shear to tear the seam apart. Optionally, connecting seam structure(s) may be the same component(s) as longitudinal support wires.

A peel away expandable introducer sheath may have two seams as shown in FIG. 5. In this exemplary embodiment a first pull tab 107a is connected to a first longitudinal half 162 of the hub 101 and a second pull tab 107b is connected to a second half 163 of the hub. Optionally, both pull tabs may be provided in a closed configuration mated to one another as shown in FIG. 2 and may be temporarily connected to one another with mating features such as a snap fit protrusion 164 and indentation 165 or magnets or other mating feature. In this configuration the second pull tab 107b wraps around a portion of the hub 101 holding it together. To open the peel away expandable introducer sheath, the second pull tab 107b is pried apart from the first pull tab 107a. While holding the first tab with one hand and the second tab with the other hand the two pull tabs may be pulled in opposite directions which opens the hub and applies shear force to the seams 160 to open the elongated tube 120 into two pieces.

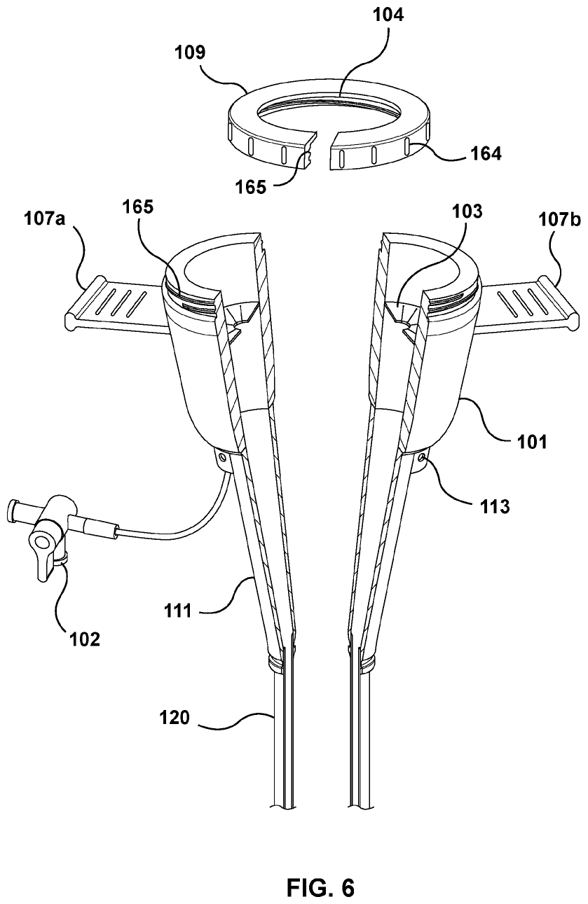

Alternatively, each of the two pull tabs 107a, 107b may be positioned on their respective hub half, for example as shown in FIG. 6. The pull tabs may be oriented in a transverse plane to the longitudinal axis.

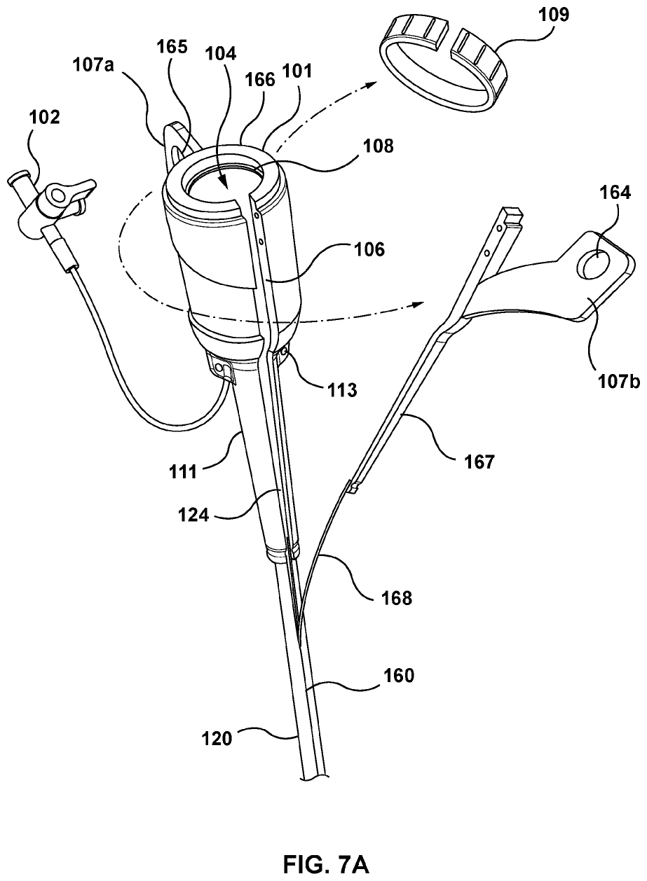

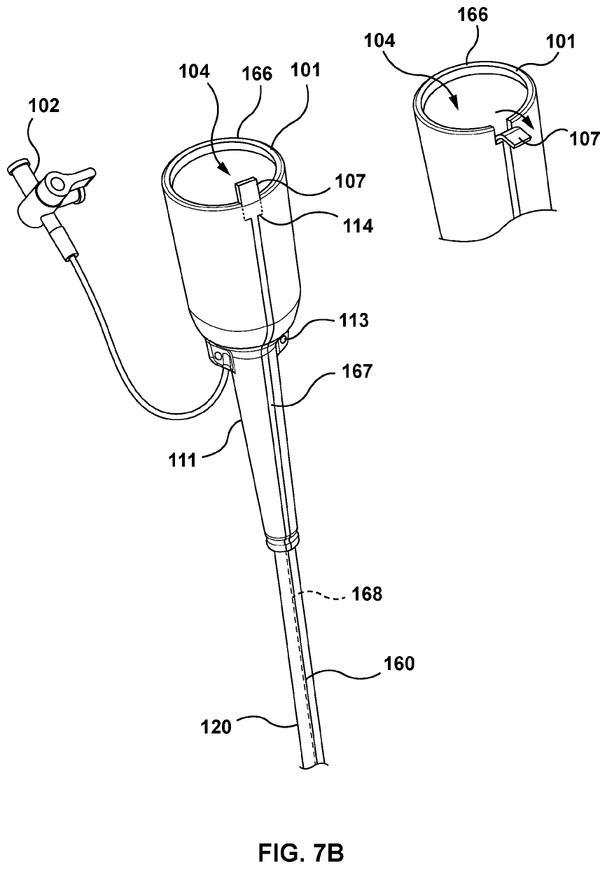

In any embodiments the introducer may optionally have a locking feature that holds the hub closed (e.g., holding two halves of a hub together, or holding an opening wedge closed, or holding the pull tabs together), which may be in the form of a removable cap 109 (see FIGS. 6 and 7A) that fits on the proximal end of the hub 101 and may fit over the parts of the hub that come apart such as the two halves (FIG. 6) or the hub and removable wedge (FIG. 7A). The removable cap 109 may fit on the hub, for example with a snap fit, friction fit, screw thread 165, and may have a textured surface 164 to facilitate handling. The cap 109 may have a radial opening to at least one side for releasing the shaft of a medical device.

Alternatively, a peel away expandable introducer sheath may have one seam as shown in FIG. 7A. In this exemplary embodiment a first pull tab 107a is connected to main body of the hub 101 and a second pull tab 107b is connected to a removable wedge 167. As shown in FIG. 7A the pull tabs may be oriented in a sagittal plane to the longitudinal axis and may be provided connected to one another wherein the second pull tab can be peeled apart from the first pull tab when the user is ready to open the introducer. The removable wedge 167 may be connected to a seam puller 168 that is imbedded in the seam 160 of the elongated tube 120. When the seam puller 168 is pulled away from a central axis of the introducer a shear force is applied to the seam 160 to open it creating an opening 124.

An alternative configuration of a peel away expandable introducer sheath having a seam through the hub 101 and elongated tube 102 is shown in FIG. 7B. Optionally, the introducer may have more than one seam (e.g., two) of a similar configuration. A pull tab 107 is a part of the hub 101 that can be snapped off, for example by bending away from a pivot point 114 to apply a shear force to material connecting the pull tab to the hub. The pull tab may release a removable wedge 167 of the hub 101, which may be connected to a seam puller 168. Optionally, the pull tab 107 may be connected to a seam connector wire 169 that holds a seam together in some embodiments.

Alternatively, a peel away expandable introducer sheath may have one or more seams 160 that pull apart the elongated tube 120 but leave the hub 101 intact, as shown in FIG. 8A. The elongated tube 120 may be peeled apart by applying a shear force to the seam by pulling pull tabs 107a and 107b radially away from the central axis of the introducer. The intact hub 101 may remain on the shaft 301 of the medical device. If a replacement introducer needs to be advanced back into the natural lumen it may be advanced through the intact hub, optionally for example through an insertion tool positioned in the introducer hub and wherein the replacement introducer sheath may lock into the insertion tool for stability and hemostasis. Optionally as shown in FIG. 8B, the insertion tool itself may remain in the intact introducer hub 101, and the insertion tool may have an insertion tool elongated tube that may be used as a repositioning sheath by advancing it into the patient's natural lumen. This configuration may be especially useful if the intact hub 101 contains one or more hemostasis valves that are not configured to be pulled apart longitudinally. A method of using the introducer of FIGS. 8A and 8B with an illustrative example in context of placing an MCS devices in the patient, may include the following steps:
Deliver a peel away expandable introducer sheath into a patient's natural lumen (e.g., into a femoral, subclavian, axillary artery through an arteriotomy), for example using a commonly practiced Seldinger Technique;
Deliver a medical device (e.g., MCS device) 300 through the peel away expandable introducer sheath 100 wherein the medical device is provided in or first inserted into an insertion tool 115;
Mate or connect the insertion tool handle 117 with the introducer hub 101;
If desired, the introducer's elongated tube 120 may be removed while the medical device (e.g., MCS device) 300 remains in the patient and a shaft 301 of the medical device remains passing through the patient's natural lumen (e.g. vasculature) and out of the body through an opening (e.g., arteriotomy), with the following steps:
While holding the medical device shaft 301 stationary with respect to the patient so the medical device 300 remains in a target location, slide the introducer's elongated tube 120 out of the patient's lumen (compression may be applied to an arteriotomy to control bleeding);
If the introducer's elongated tube 120 is held together with a seam connector 169, pull the seam connector(s) out of the seam(s) 160;
Peel the introducer's elongated tube 120 apart by applying shear to the at least one seam 160 by pulling pull tabs 107a and 107b apart from one another and radially away from the introducer's central axis; This also separates the elongated tube 120 and optionally the strain relief 111 from the introducer hub 101 while leaving the hub and connected insertion tool 115 remaining around the device's shaft 301, wherein the elongated tube 116 of the insertion tool extends distally from the introducer hub 101;
Insert the insertion tool's elongated tube 116 into the patient's lumen (e.g., into the arteriotomy) over the device's shaft 301, optionally with an insertion tool dilator, if an introducer sheath is desired for repositioning or removing the medical device 300, or for performing other interventions through the patient's endoluminal access.

Alternatively, instead of inserting an insertion tool's elongated tube into the patient's lumen (e.g., arteriotomy), for example if an insertion tool is not used or is not configured for introduction into the patient, a separate repositioning introducer sheath may be used. For example, the medical device may be advanced through the separate repositioning introducer that remains out of the body on the medical device shaft 301 before advancing the medical device through the peel away expandable introducer sheath 100. If the peel away expandable introducer sheath is removed and peeled apart. The repositioning introducer may be advanced over the shaft 301 into the patient's lumen (e.g., arteriotomy) if needed. In an example where the peel away expandable introducer has a hub 101 that remains intact (e.g., as in the embodiment shown in FIG. 8A) the repositioning introducer may be advanced through the intact hub 101 before advancing into the patient.

### Examples of Elongated Tubes and Seams

A variety of exemplary embodiments of peel away expandable introducer sheaths are further disclosed herein. It is understood that features and components disclosed herein and associated with a particular embodiment may be combined in various ways to create alternative embodiments of peel away expandable introducer sheaths. It is also understood that some features relevant for peeling an introducer sheath open may be relevant for novel peel away introducers that are not necessarily expandable and conversely, some features relevant for expandable introducer sheaths may be relevant for novel expandable introducers that are not necessarily configured for peeling apart.

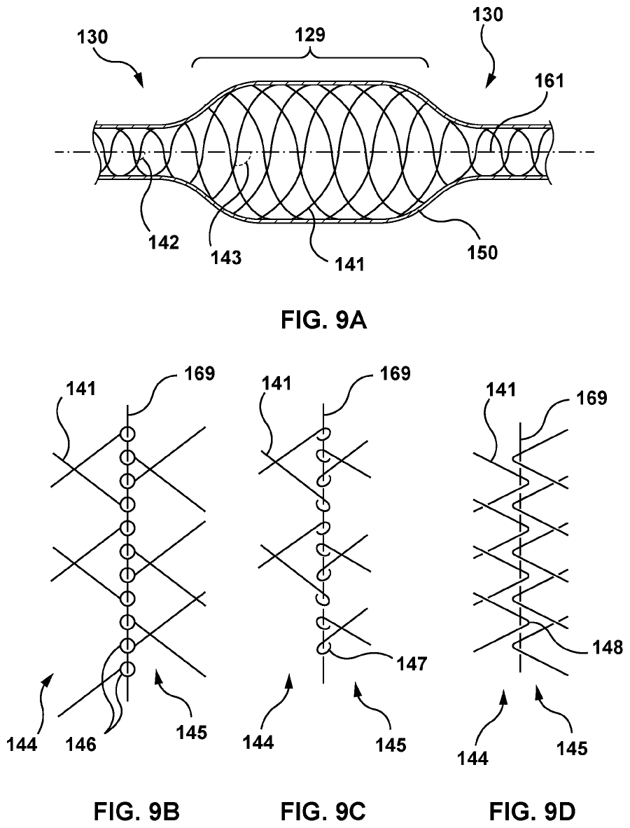

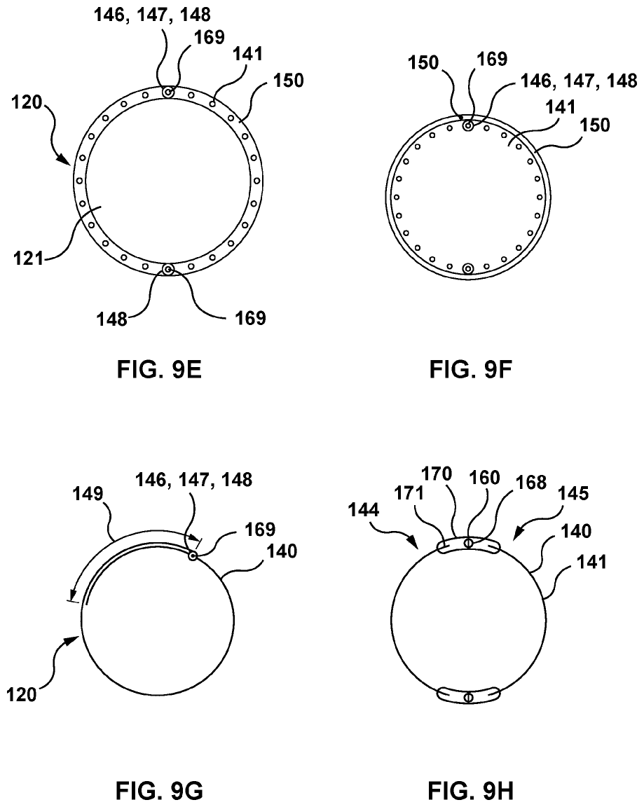

FIG. 9A is a schematic illustration of an exemplary embodiment of a peel away expandable introducer sheath wherein the elongated tube 120 is made from a structural layer 140 made from braided or woven fibers 141. The material of the fibers may be a material that is flexible and elastic yet has sufficient rigidity to meet its functional requirements with a cross sectional thickness in a range of 0.003″ to 0.010″. For example, the fibers may be made from superelastic Nitinol, spring stainless steel, or a polymer. The structural layer made from braided, woven, or knitted (or a combination thereof) fibers 141 may expand radially, for example by allowing movement in the fibers wherein at least the portion 129 of the elongated tube 120 in which inner radial force is applied has fibers crossing at an increased braid angle 143 compared to the neutral braid angle 142 in portions of the elongated tube 130 that are in neutral state. The elongated tube also has an expandable membrane 150, which may be bonded to the fibers 141, for example via dip coating, over coating, substrate bonding or adhesive. Alternatively, an expandable membrane may be a coaxially aligned tube positioned over the outer surface of the structural layer 140. Optionally, the elongated tube may include one or more (e.g., two, three, four) longitudinal support wires 161 in the wall 125 of the elongated tube. The braided, woven, or knitted fibers 141 may be held in a tubular shape by one or two seams 160. In one example the seam 160 consists of a seam connector wire 169 holding two sides of the braided, woven, or knitted structure together, wherein removal of the seam connector wire 169 disconnects the sides of the braided, woven, or knitted structure allowing them to be separated. The seam connector wire(s) 169 may be pulled out of the introducer from the proximal end 105 of the introducer. For example, the wire(s) 169 may be connected to a pull tab or ring 172 accessible from the hub 101 (see FIG. 5), which optionally may be contained in closed pull tabs 107 and revealed when the pull tabs 107 are opened.

As shown in FIG. 9B a first side 144 of the braided or woven structure is held to the second side 145 by a seam connector wire 169 threaded through rings 146 connected (e.g., laser welded) to the ends of each fiber 141. Alternatively, as shown in FIG. 9C, the ends of the fibers may be formed into hooks 147 through which a seam connector wire 169 is threaded. Alternatively, as shown in FIG. 9D, the fibers do not terminate in ends at the seam but instead form closed loops 148 through which a seam connector wire 169 is threaded. Optionally, as shown in FIG. 9E, the membrane 150 may be bonded to the seam connector wire 169 so that when the wire 169 is removed it also tears the membrane allowing the first side 144 to be separated from the second side 145. Alternatively, as shown in FIG. 9F, the seam connector wire 169 may not be bonded to the membrane, for example it may be masked during a bonding step, which may facilitate easier removal of the seam connector wire 169, and the membrane may be cut by the user for example with a safety cutting tool, or it may be opened by pulling on a seam puller wire 168 embedded in a seam of the membrane. Note that FIGS. 9E and 9F show two seams, but alternatively an elongated tube may have one seam. Optionally, as shown in FIG. 9G, the wall 125 of the elongated tube 120 may have a structural layer 140 (e.g., made from braided or woven fibers) that overlaps on itself for at least a portion 149 (e.g., <=20%, <=30%, <=40%, <=50%, <=100%) of the circumference, which may improve hoop strength of the structural layer which in turn may improve functional characteristics.

An alternative seam is shown in cross sectional FIG. 9H, wherein the woven, braided, or knitted fibers 141 form at least a portion of the structural layer 140 and a portion 171 of the fibers are embedded (e.g., via over-molding) into a polymeric longitudinal seam member 170, which adds to creating hoop strength, connects a first side 144 to a second side 145, and is adapted to be separated for example by pulling a seam puller 168 to apply shear to the polymer seam member 170. The one or more seams 160 may be aligned on the wall of the elongated tube parallel to the axis of the tube or alternatively the seam may follow a helical path around and along the length of the elongated tube.

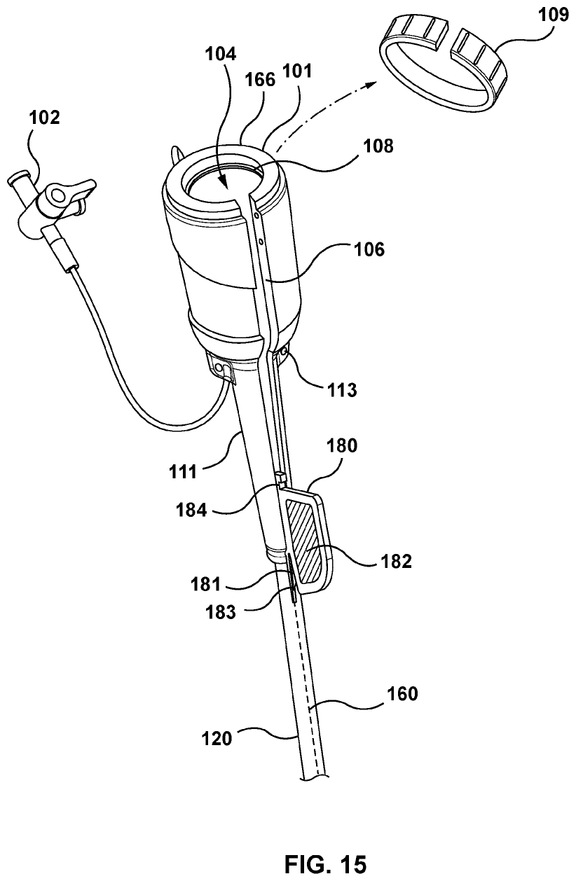

An alternative seam and device for separating the seam is shown in FIG. 15, wherein a cutting blade 181 is held in a seam cutting tool 180 that may be used to cut open the seam 160 when a user wishes to open the introducer. The cutting tool 180 is adapted to connect to the hub 101. The blade 181 is held in a recess between a tongue 183 and a handle 182. The tongue 183 is arranged in an inner lumen of the elongated tube 120 and is adapted to slide between a device shaft 301 and a seam 160 of the elongated tube. For example, the tongue 183 may have a maximum thickness that fits between the device shaft and the seam (e.g., in a range of 0.08 to 0.25 mm). The tongue 183 may have a shape or curvature that fits in the space between the device shaft and the seam such as a partial cylinder or a curvature with a radius of curvature similar (e.g., slightly smaller, within 5%) to the radius of curvature of the elongated tube 120. The tongue 183 may have rounded edges and lack sharp edges, which may prevent the tongue from getting snagged on the elongated tube 120. The tongue 183 is adapted to slide under the wall of the elongated tube 120 and guide the seam 160 into the blade 181 as a user grasps the handle 182 and slides it distally. The blade 181 may be held at an angle (e.g., an obtuse angle) to the axis of the elongated tube 120, which may facilitate cutting of the seam 160. The handle 182 in part defines the recess in which the blade 181 is held and may have a textured surface to provide grip when grasping. Optionally, the cutting tool 180 may be adapted to be held firmly in a side of the hub 101 until released. For example, the cutting tool 180 may have a tab 184 that mates with a locking cap 109 to prevent the cutting tool 180 from moving. When the locking cap 109 is removed (e.g., unscrewed) the tab 184 may be released. Optionally, the hub 101 may have a hub seam and the strain relief 111 may have a strain relief seam that are opened by tearing a seam tab, which may also release the cutting tool 180. Optionally, the introducer may have more than one seam 160 (e.g., two) of a similar configuration, which can be cut open using one or multiple cutting tools 180.

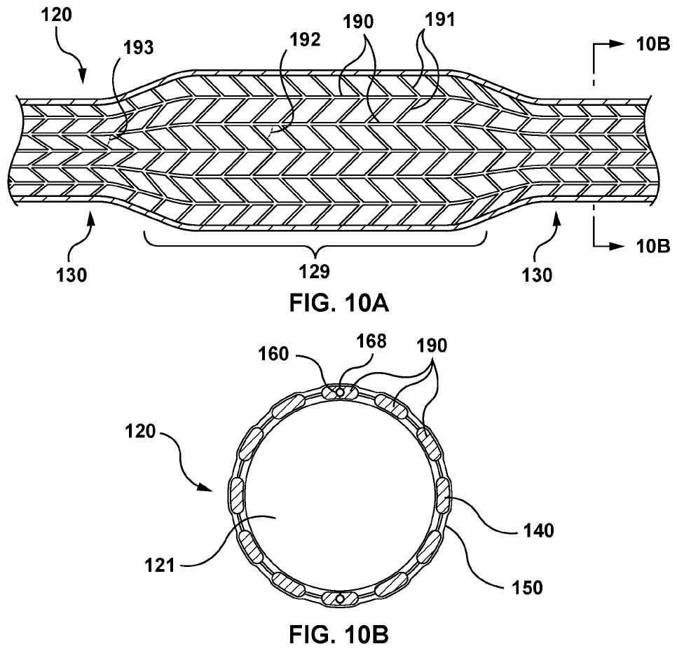

An alternative exemplary embodiment of an elongated tube 120 is shown in FIGS. 10A and 10B, wherein the structural layer 140 is formed by a laser cut tube (e.g., superelastic Nitinol or flexible polymer having a wall thickness in a range of 0.003″ to 0.010″) and has longitudinal members 190 connected to adjacent longitudinal members by angled cross members 191. The space between two adjacent longitudinal members may be referred to as a row. Within a row all angled cross members are angled in the same direction and the direction of the angled cross members alternates in adjacent rows. In an expanded section 129 of the elongated tube an angle 192 between angled cross members and longitudinal members 190 is greater than an angle 193 in the neutral state 130 of the elongated tube. As shown in the cross-sectional FIG. 10B the structural layer 140 may be embedded in a membrane 150, for example, via dip coating. Elastic properties of the membrane may cause the elongated tube to assume its neutral state when radial force is removed from the lumen 121. A seam 160 in this embodiment may be formed by embedding a seam puller 168 in one or more (e.g., two opposing) longitudinal members 190, wherein pulling the seam puller 168 to the side applies shear to tear open the longitudinal member. If the laser cut tube is Nitinol one or more polymer longitudinal members 190 containing a seam 160 may be made by cutting a longitudinal slit in the tube and molding a polymer longitudinal member with a seam into the slit.

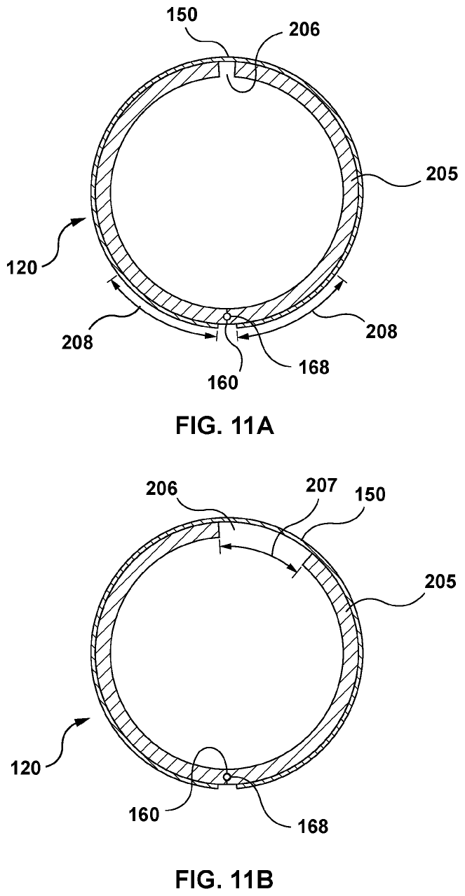

An alternative embodiment of an elongated tube 120 is shown in neutral state in FIG. 11A, and expanded state in FIG. 11B, wherein the structural layer is formed from a polymer tube 205 (e.g., Pebax®) that is radially expandable along more or more slits 206. The elastic membrane 150 stretches to allow the slit 206 to open to an increased gap 207 during expansion and causes the elongated tube 120 to assume its neutral state when radial force is removed from the inner surface. The stretchable membrane 150 covers the slit 206 and stretches to cover the expanded gap 207 to form a liquid seal and to prevent tissue from getting pinched in the gap 207. The membrane 150 may be bonded at only a portion 208 of the outer surface of the structural layer allowing the unbonded portion of the membrane to elongate during expansion. As shown, the membrane 150 may be non-continuous in circumference and have a longitudinal gap aligned with a seam 160 in the polymer tube 205. A seam puller 168 may be embedded in the polymer tube wall at the seam 160.

### Distal End

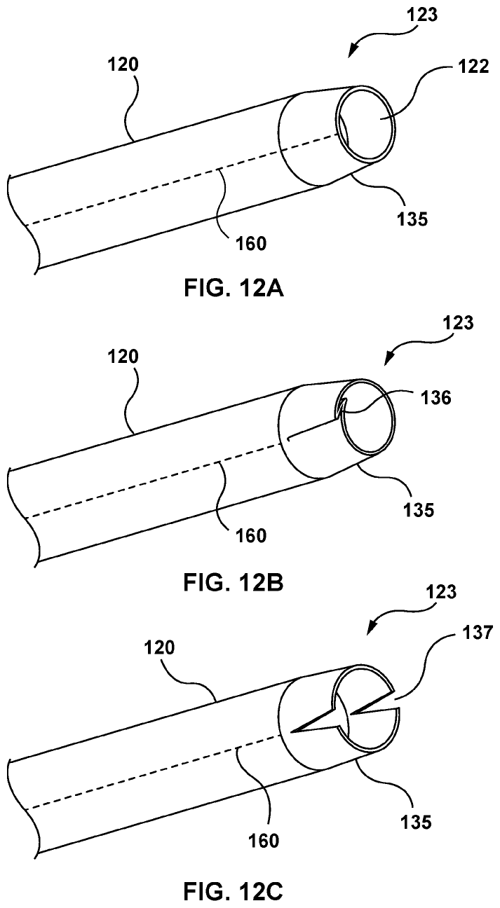

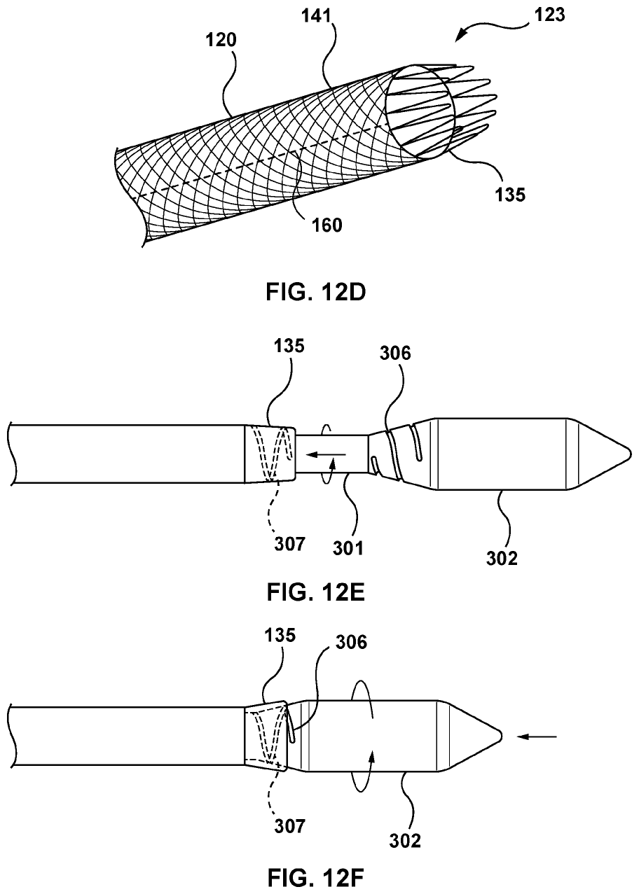

The distal end 123 of the elongated tube 120 has a distal opening 122 and optionally has an expandable atraumatic distal tip 135 that less rigid than the remainder of the elongated tube to reduce a risk of iatrogenic injury, which a more rigid elongated tube is at risk of causing. The expandable atraumatic distal tip may be made from a soft durometer material, optionally stretchable, and be absent the structural layer 140. Optionally, the expandable distal tip may be made from the stretchable membrane material and may be manufactured during the step of applying the stretchable membrane 150 (see FIG. 12A). Optionally the expandable atraumatic distal tip is distally tapered. Optionally the expandable atraumatic distal tip has one or more pleats 136 to facilitate expansion when inner radial force is applied (see FIG. 12B). Alternatively and optionally, the expandable atraumatic distal tip has one or more wedge shaped cutouts 137 to facilitate expansion and also to reduce force needed to retract a large part of a medical device 302 back into the elongated tube and out of the body. The cutouts 137 may be aligned with the seam 160 to facilitate opening of the expandable atraumatic distal tip when the seam is opened (see FIG. 12C).

Optionally, the fibers 141 used to form the structural layer 140 may extend into the expandable atraumatic distal tip 135 as shown in FIG. 12D. For example, the fibers 141 may form closed end loops in within the distal tip 135. Optionally, the fibers may be made with a shape memory material such as Nitinol and be shape set to have a distally inward taper as shown or alternatively have a distally outward taper that is held in an inward taper configuration by a dilator. Alternatively and optionally, the expandable atraumatic distal tip has a helical groove or thread 307 adapted to mate with a helical groove or thread 306 on a proximal end of a large part of a medical device 302. In use, while retracting the large part of the medical device 302 into the expandable atraumatic distal tip 135 rotation of the shaft 301 of the medical device may be transmitted to the large part 302 and the threads or grooves 306 on the large part 302 may engage with the treads or grooves 307 of the expandable atraumatic distal tip (see FIG. 12E). The threads or grooves 306 on the large part 302 may follow a funnel shaped contour on the proximal end of the large part 302. As the large part 302 is rotated and the threads and grooves engage the expandable atraumatic distal tip is expanded and the large part of the medical device 302 is pulled into the elongated tube (see FIG. 12F), which may reduce pull force required to remove the medical device through the introducer sheath.

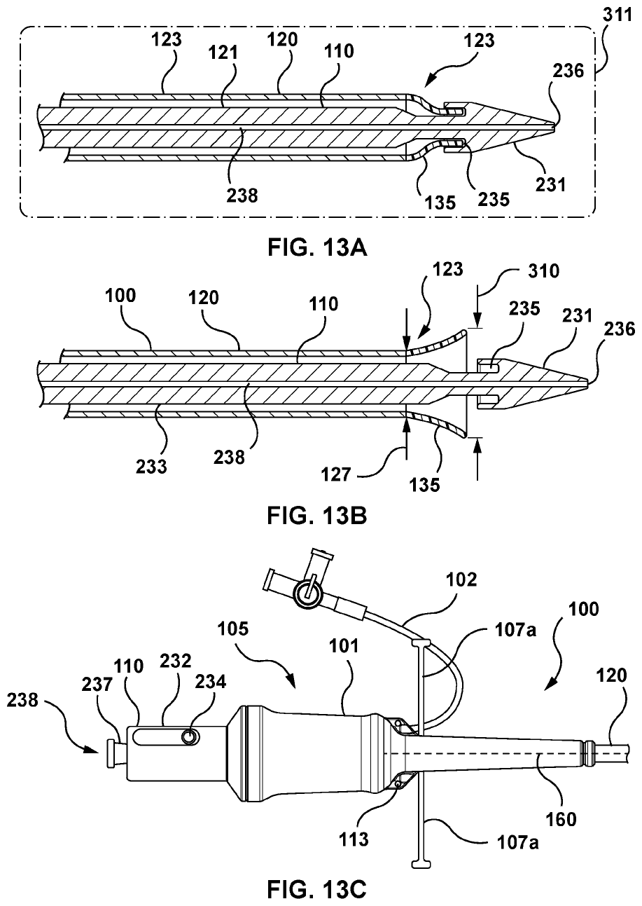

In an alternative embodiment of an expandable atraumatic distal tip 135, the expandable atraumatic distal tip 135 may be adapted to take a delivery configuration when constrained by a distal tip 231 of a dilator 110, as shown in FIG. 13A and automatically transition to an expanded funnel shape, as shown in FIG. 13B, when the constraint of the dilator tip 135 is removed. The expanded funnel shape may have a distal diameter 310 that is greater than the neutral state outer diameter 127 of the elongated tube. As an illustrative example for an introducer intended for delivery though a femoral artery, the distal diameter 310 may be intended for placement in an ascending aorta and be small enough to permit sufficient blood flow through the aorta. For example, the diameter of the elongated tube 127 may be in a range of 8F to 11F and the distal diameter of the funnel shaped tip 310 may be in a range of 1F to 4F greater than the diameter of the elongated tube. The tip 135 may be held in a contracted shape by a dilator during insertion into a body lumen and removal of the constraint will cause the tip to expand into its neutral funnel shape. This may facilitate removal of a large part 302 of a medical device by reducing pull force and accepting the medical device without buckling and with reduced friction. The distal tip 231 of the dilator 110 may have a conical shape for dilating an arteriotomy or other entry point and may have a depression 235 in which the expandable atraumatic distal tip is constrained in the delivery configuration.

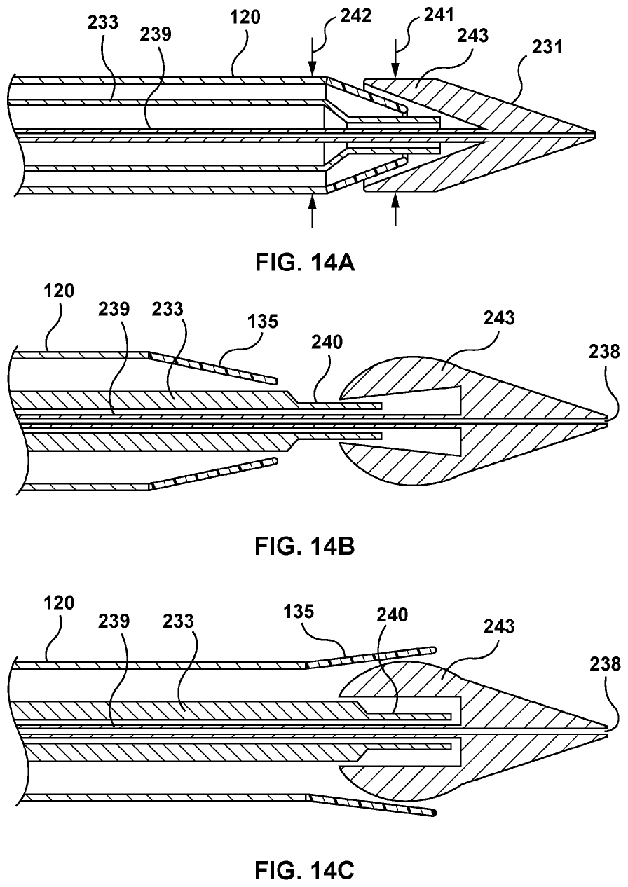

Another embodiment of an introducer and dilator system for facilitation of removal of a medical device is shown in FIGS. 14A, 14B and 14C. FIG. 14A shows a delivery configuration wherein the dilator tip 231 has a conical shape and an outer diameter 241 that may be in a range equal to the inner diameter of the elongated tube 120 to the outer diameter 242 of the elongated tube 120. The dilator tip 231 has a recess 235 that holds the distal tip 135 in a narrow delivery configuration. The recess 235 may be defined by a collapsible portion 243 of the dilator tip 231. The collapsible portion 243 may elastically collapse when the dilator is advanced with respect to the distal tip 135 as shown in FIG. 14B. The collapsible portion 243 may be made from an elastic material (e.g., silicone) that predisposes the collapsible portion to a collapsed configuration when unconstrained or when the distal tip 135 is not holding it open. When the dilator is retracted and the dilator tip 231 enters the introducer sheath, the collapsible portion 243 enters the inside of the introducer's distal tip 135 opening it into a funnel shape such that distal end of the distal tip 135 has an inner diameter equal to the outer diameter 241 of the dilator tip 231, as shown in FIG. 14C. Optionally, the dilator's distal tip 231 may be connected to a rod 239 that is slidably movable with respect to a dilator shaft 233 so the dilator tip 231 can be advanced with respect to the dilator shaft 233. The rod 239 may have a guidewire lumen running through it. The dilator shaft may have a narrowed diameter 240 on its distal end. By adjusting the position of the rod 239 with respect to the shaft 233 the collapsible portion 243 may be collapsed onto the narrowed diameter 240 or the shaft 233 which may adjust the outer diameter 241 of the dilator tip 231 as shown in FIGS. 14B and 14C. The narrower configuration (FIG. 14B) may facilitate getting the collapsible portion 243 into the introducer's distal end 135 or removal of the dilator through the elongated tube 120. The larger configuration (FIG. 14C) may be used to enlarge the introducer's distal tip 135.

FIG. 13C shows a proximal end 105 of the peel away expandable introducer sheath 100 with the dilator 110 positioned in the introducer with the dilator handle 232 locked (e.g., snap fit, friction fit, threaded) into the hub 101 of the introducer 100. Optionally, the dilator shaft 233 can be extended distally relative to the handle 232 by manipulating an actuator 234 on the handle 232 to disengage the depression 235 from the introducer's distal tip 135. Optionally, the dilator may have a guidewire lumen 238 passing through the length of the dilator from a proximal guidewire port 237 to a distal tip guidewire port 236.

Optionally, a peel away expandable introducer 100 may be provided in a kit along with a dilator 110. The kit may further contain other components used in a catheterization procedure such as guidewires, introducer needles, diagnostic catheters, or interventional medical devices. As shown in FIG. 13A and introducer 100 having a distal end 135 adapted to assume a funnel shape may be provided in a kit 311 with a dilator 110 positioned in the introducer's lumen 121 with the dilator tip 231 engaged with the introducer distal end 135 to hold it in the delivery configuration. The dilator handle 232 may be locked into the introducer hub 101.

Optionally, any embodiment of an expandable atraumatic distal tip has a low friction inner layer, coating or surface.

While the above description provides examples of one or more processes or apparatuses, it will be appreciated that other processes or apparatuses may be within the scope of the accompanying claims.

Specific embodiments described herein are not intended to limit any claim and any claim may cover processes or apparatuses that differ from those described herein, unless specifically indicated otherwise. The claims are not limited to apparatuses or processes having all of the features of any one apparatus or process described herein or to features common to multiple or all of the apparatuses described herein, unless specifically indicated otherwise. It is possible that an apparatus or process described herein is not an embodiment of any exclusive right granted by issuance of this patent application. Any subject matter described herein and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1-65. (canceled)

66. A method of introducing a medical device through an introducer sheath, comprising the steps of:
providing an expandable, peel away introducer sheath having a proximal end, a distal end, and a tubular side wall defining a central lumen selectively enlargeable from a relaxed inside diameter to an enlarged inside diameter;
positioning the introducer sheath into a vessel of a patient while in a relaxed inside diameter configuration;
providing a catheter carrying a medical device which has an outside diameter that is larger than the relaxed inside diameter;
advancing the medical device distally through the lumen causing an expansion zone of the side wall in the vicinity of the medical device to momentarily reach the enlarged inside diameter to accommodate passage of the medical device; and thereafter
splitting the introducer sheath axially to create a longitudinal opening and expose the lumen to permit lateral removal of the introducer sheath from the catheter.

67. A method as in claim 66, wherein the expansion zone progressively advances distally along the tubular side wall in response to distal advance of the medical device.

68. A method as in claim 67, wherein the medical device comprises a mechanical circulatory support device.

69. A method as in claim 66, wherein splitting the introducer sheath comprises pulling on at least one pull tab to create the longitudinal opening.

70. A method as in claim 66, further comprising the step of coupling an insertion tool to the proximal end prior to the advancing step.

71. A method as in claim 69, wherein splitting the introducer sheath comprises pulling on two opposing pull tabs to axially split the introducer sheath into two pieces.

72. A method as in claim 70, wherein the insertion tool comprises an extension tube configured to be advanced into the patient.

73. A method as in claim 72, further comprising a step of advancing the extension tube of the insertion tool into the vessel of the patient following a removal of the introducer sheath from the catheter.

74. A method as in claim 66, wherein creating the longitudinal opening occurs in a hub on the proximal end of the introducer sheath.

75. A method as in claim 74, wherein the longitudinal opening is formed by splitting the hub axially into two pieces.

76. A method as in claim 75, further comprising a removable retainer for retaining the two pieces together.

77. A method as in claim 74, wherein the longitudinal opening is formed by splitting the hub axially into two halves.

78. A method as in claim 77, further comprising a pull tab on each half.

79. A method as in claim 78, wherein a first pull tab on a first half wraps around at least a portion of the hub connecting the first half to a second half.

80. A method as in claim 79, wherein the first pull tab removably mates with the second pull tab on the second half.

81. A method as in claim 74, further comprising a step of engaging an accessory device to a locking mechanism on the hub.

82. A method as in claim 81, wherein the accessory device comprises a dilator and wherein the dilator is configured to releasably hold an expandable atraumatic distal tip in a compressed state.

83. A method as in claim 82, wherein the dilator comprises a handle with an actuator configured to release the expandable atraumatic distal tip from the compressed state.

84. A method as in claim 66, wherein the introducer sheath further comprises a radially expandable structural layer comprising braided, woven, or knitted fibers.

85. A method as in claim 84, wherein the structural layer overlaps on itself for at least a portion of a circumference of the introducer sheath.
